(19) United States
(12) Patent Application Publication
(10) Pub. No.: US 2007/0016052 A1
(43) Pub. Date: Jan. 18, 2007

(54) Ultrasonic diagnostic apparatus

(75) Inventor: Hiroshi Fukukita, Setagaya-ku (JP)

Correspondence Address: HAMRE, SCHUMANN, MUELLER & LARSON P.C., P.O. BOX 2902-0902, MINNEAPOLIS, MN, 55402, US

(73) Assignee: MATSUSHITA ELECTRIC INDUSTRIAL CO., LTD., Osaka (JP)

(21) Appl. No.: 10/567,944

(22) PCT Filed: Sep. 22, 2004
(86) PCT No.: PCT/JP04/14293

§ 371 Date: Feb. 10, 2006

(30) Foreign Application Priority Data
Sep. 24, 2003 (JP) 2003-331961

(51) Int. Cl. A61B 8/14

(52) U.S. Cl. 600/459

(57) ABSTRACT

A two-dimensional array including two-dimensionally arranged transducers with M rows (M=4) and N columns (N=12) is separated into eight sub-arrays each including six transducers with m rows (m=2) and n columns (n=3). The sub-arrays with J rows (J=2) and K columns (K=4) are connected to eight intra-group processors IP(JJ, KK) (JJ=1 to 2, KK=1 to 4), respectively. A switch allows the selection of j pieces in the row direction (j=2) (j≦J) and k pieces in the column direction (k<K) from the intra-group processors IP(JJ, KK) while shifting the selection in the column direction. The number of signal lines included in a cable for the connection with a main unit can be reduced, and power consumption also can be suppressed.

### TECHNICAL FIELD

The present invention relates to an ultrasonic diagnostic apparatus having a two-dimensional array in which a plurality of electroacoustic devices (hereinafter simply referred to as transducers) are arranged two-dimensionally for scanning a subject three-dimensionally.

### BACKGROUND ART

A conventional ultrasonic diagnostic apparatus, as shown in FIG. 8, is composed of a two-dimensional array 102 in which a plurality of transducers 101 are arranged two-dimensionally and intra-group processors IP(J, K) (J=1, 2, K=1, 2) that are connected with sub-arrays of the transducers 101 with two rows and two columns. The intra-group processors IP(J, K) are connected with a control unit 104 of a main unit 107 via a cable 108.

A reception signal from a sub-array undergoes beam-forming by the intra-group processor IP(J, K), which further undergoes beam-forming by a delay addition unit (not illustrated) in the control unit 104. Practically, 3,000 pieces of transducers and 120 pieces of intra-group processors are provided, for example, whose power consumption is 2 watts in total, and at least 120 signal lines are included in the cable 8 (for example, see JP 2000-33087 A, pages 3 and 10 to 11, FIG. 3).

In such a conventional ultrasonic diagnostic apparatus, in the case of having a large number of transducers (N=200) in a longer axis direction (column direction) as in a convex array, if such transducers are separated (M=60) in a shorter axis direction (row direction) so as to configure a two-dimensional array, the required number of the transducers increases to as large as 12,000, which is four times that of the example shown in FIG. 8. Therefore, the number of the intra-group processors also increases to 480, which is four times that of the example. Consequently, the power consumption increases to 8 watts, thus causing the problem of heat generation. Moreover, since at least 480 signal lines are included in the cable, such a thick cable is difficult to handle.

### DISCLOSURE OF INVENTION

In order to solve the above-stated conventional problems, it is an object of the present invention to provide an ultrasonic diagnostic apparatus including selectively operable intra-group processors for reducing the number of signal lines included in a cable for the connection with a main unit and suppressing power consumption.

In order to achieve the above-stated object, an ultrasonic diagnostic apparatus of the present invention includes: an electroacoustic conversion unit including electroacoustic conversion devices with M rows and N columns, in which sub-arrays are arranged at least two-dimensionally with J rows and K columns, each of the sub-arrays including electroacoustic conversion devices with m rows and n columns, where M=m×J, N=n×K; intra-group processors with J rows and K columns provided corresponding to the respective sub-arrays; and a selection unit that selects intra-group processors with j rows (j≦J) and k columns (k<K) as a target from the intra-group processors with J rows and K columns, the selection being performed while shifting the selection target of the intra-group processors in a column direction.

With this configuration, the intra-group processors can operate selectively, so that the number of signal lines included in a cable for the connection with a main unit can be reduced, and power consumption also can be suppressed.

In the ultrasonic diagnostic apparatus of the present invention, the selection unit may select intra-group processors with j rows and k columns as the target while shifting the selection target of the intra-group processors in a row direction. With this configuration, the number of signal lines included in a cable for the connection with a main unit further can be reduced, and power consumption also further can be suppressed.

In the ultrasonic diagnostic apparatus of the present invention, the selection unit may include a reception switch that selectively connects a reception signal from the intra-group processors with a reception beam former. With this configuration, the number of signal lines included in a cable for the connection with a main unit can be reduced.

In the ultrasonic diagnostic apparatus of the present invention, the selection unit may include a data switch that selectively supplies group focus data to the intra-group processors. With this configuration, the amount of group focus data supplied to the intra-group processors can be reduced.

In the ultrasonic diagnostic apparatus of the present invention, the selection unit may include a power supply switch that selectively supplies a group power supply to the intra-group processors. With this configuration, power consumption of the intra-group processors can be reduced.

In the ultrasonic diagnostic apparatus of the present invention, the selection unit may include a clock switch that selectively supplies a clock signal to the intra-group processors. With this configuration, power consumption of the intra-group processors can be reduced.

### BRIEF DESCRIPTION OF DRAWINGS

FIG. 1 is a block diagram showing the configuration of an ultrasonic diagnostic apparatus according to Embodiment 1 of the present invention.

FIG. 2 is a schematic diagram for explaining an operation of a two-dimensional array of FIG. 1.

FIG. 3 is a block diagram showing one example of the configuration of a major part of an ultrasonic diagnostic apparatus according to Embodiment 2 of the present invention.

FIG. 4 is a block diagram showing one example of the configuration of a major part of an ultrasonic diagnostic apparatus according to Embodiment 3 of the present invention.

FIG. 5 is a block diagram showing one example of the configuration of a major part of an ultrasonic diagnostic apparatus according to Embodiment 4 of the present invention.

FIG. 6 is a block diagram showing one example of the configuration of a major part of an ultrasonic diagnostic apparatus according to Embodiment 5 of the present invention.

FIG. 7 is a block diagram showing one example of the configuration of a major part of an ultrasonic diagnostic apparatus according to Embodiment 6 of the present invention.

FIG. 8 is a block diagram showing the configuration of a conventional ultrasonic diagnostic apparatus.

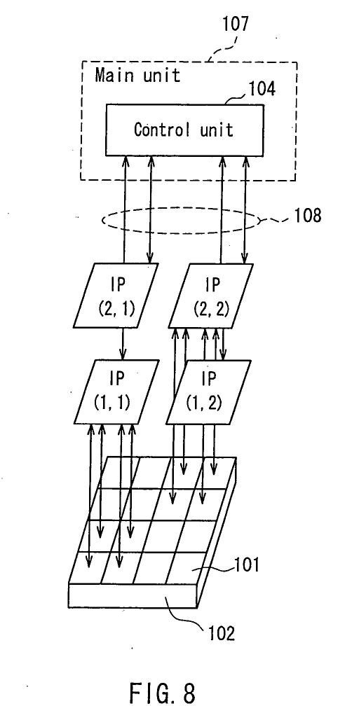

### DESCRIPTION OF THE INVENTION

The following describes preferred embodiments of the present invention, with reference to the drawings.

### Embodiment 1

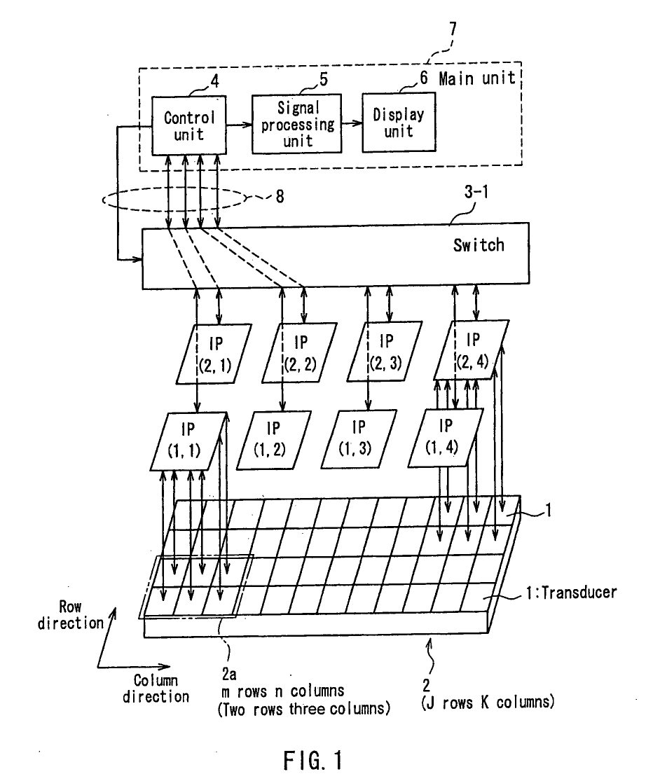

FIG. 1 is a block diagram showing the configuration of an ultrasonic diagnostic apparatus according to Embodiment 1 of the present invention.

In FIG. 1, transducers 1 as electroacoustic conversion devices are arranged two-dimensionally with M rows (M=4) and N columns (N=12) so as to make up a two-dimensional array 2 (electroacoustic conversion means). The two-dimensional array 2 is separated into eight sub-arrays 2a each including six transducers 1 with m rows (m=2) and n columns (n=3). Therefore, the sub-arrays 2a are in the arrangement of J rows (J=2) and K columns (K =4), where the relationships of M=m×J and N=n×K are satisfied. Each sub-array 2a is connected with one of eight intra-group processors IP(JJ, KK) (JJ=1 to 2, KK=1 to 4). The intra-groups IP(JJ, KK) are connected with a switch 3-1 (selection means).

Out of the intra-group processors IP(JJ, KK), four intra-group processors in total including j (=2) pieces in the row direction (j≦J) and k (=2) pieces (k<K) in the column direction are selected by the switch 3-1. Reception signals from the selected intra-group processors IP are supplied to a control unit 4 via signal lines in a cable 8, where the signals are delayed and added. The delayed and added signal output from the control unit 4 is supplied to a signal processing unit 5 so as to be processed as an image signal, and then an image is displayed by a display unit 6. Herein, the control unit 4, the signal processing unit and the display unit 6 make up a main unit 7.

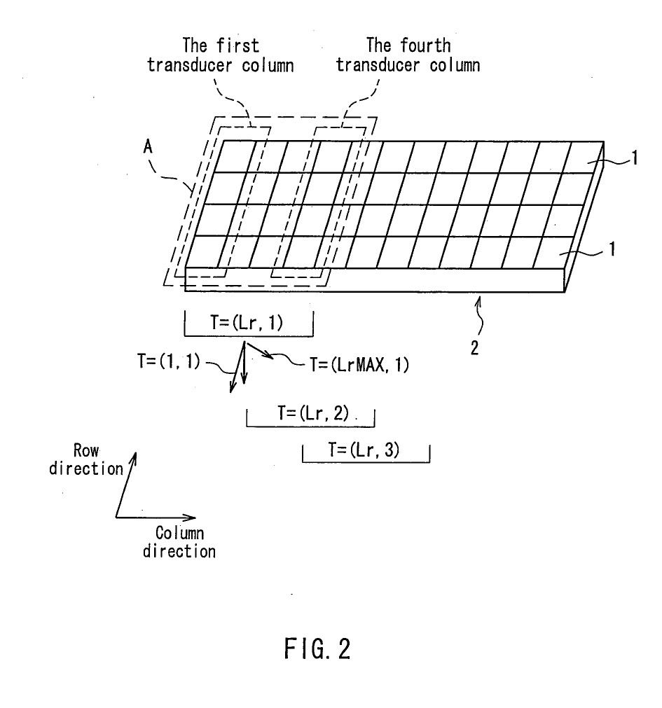

The following describes an operation of the ultrasonic diagnostic apparatus configured as above, with reference to FIG. 2 as well as FIG. 1. FIG. 2 is a schematic diagram for explaining an operation of the two-dimensional array 2 of FIG. 1.

Firstly, the control unit 4 controls the switch 3-1 to select four intra-group processors in total of JJ=1 to 2 and KK=1 to 2 from the intra-group processors IP(JJ, KK). Each of the four intra-group processors IP is connected with a sub-array 2a including the transducers 1 with 2 rows and 3 columns, so that the transducers 1 with 4 rows and 6 columns are selected. The control unit 4 sends data to the intra-group processors IP so that transducers with 4 rows and 4 columns among the transducers with 4 rows and 6 columns generate transmission pulses. In FIG. 2, the state of the transmission by the intra-group processors IP is represented with T=(Lr, Lc). Herein, Lr (1 to LrMAX) represents a direction of sector scanning in the row direction and Lc represents a selection state of the transducers 1 in the column direction. As shown in FIG. 2, in the state of T=(Lr, 1), the transducers in the first to the fourth columns are selected in the column direction to form an aperture A, and sector scanning in the row direction is performed. In the state of T=(Lr, 2), the transducers in the third to the sixth columns are selected in the column direction to form an aperture (not illustrated), and sector scanning in the row direction is performed. In this way, while the aperture is shifted at intervals narrower than the width of the sub-array 2a in the column direction, the transmission by the intra-group processors of JJ=1 to 2 and KK=1 to 2 among the intra-group processors IP(JJ, KK) are completed.

Next, the control unit 4 controls the switch 3-1 to select four intra-group processors in total of JJ=1 to 2 and KK=2 to 3 from the intra-group processors IP(JJ, KK). In the transmission state of T=(Lr, 3), the transducers in the fourth to the ninth columns are selected in the column direction to form an aperture, and sector scanning in the row direction is performed. In this way, the transmission by the intra-group processors of JJ=1 to 2 and KK=2 to 3 among the intra-group processors IP(JJ, KK) are completed.

In this way, while the transducer columns selected in the column direction are shifted, sector operation is performed in the row direction, whereby one transmission cycle by the two-dimensional array 2 is completed. In response to the above-stated transmission, reception signals are processed as follows.

In the case of the transmission state of T=(Lr, Lc), data is sent from the control unit 4 to the intra-group processors IP so that directivity of the reception by the selected four intra-group processors IP agrees with directivity of the transmission. Four reception signals undergoing beam-forming in the four intra-group processors IP are sent to the control unit 4 via the switch 3-1 and four signal lines in the cable 8. The reception signals are delayed and added by a reception beam former (not illustrated) that is included in the control unit 4, thus becoming the delayed and added signal output. In the case where the switch 3-1 is not provided, eight output signal lines for all of the intra-group processors IP have to be connected with the control unit 4. According to the present embodiment, however, the number of the output signal lines can be decreased to four. Further, the reception beam former of the control unit 4 may have a parallel reception function to have the directivity of the reception in a plurality of directions that slightly deviate from the directivity of the transmission. Thereby, the scanning in a broad range can be conducted with one transmission.

As stated above, according to the present embodiment, the switch 3-1 selects the intra-group processors with j rows and k columns from the intra-group processors with J rows and K columns, and the selection target of the intra-group processors is shifted in the column direction. Thereby, the number of the signal lines in the cable 8 that convey the reception signals from the intra-group processors to the control unit 4 can be reduced.

Note here that according to the above description, the relationship of the number j of the selected intra-group processors in the row direction and the number J of all intra-group processors in the row direction satisfies j≦J. However, j<J is possible also, where the selection of the intra-group processors is shifted in the row direction.

### Embodiment 2

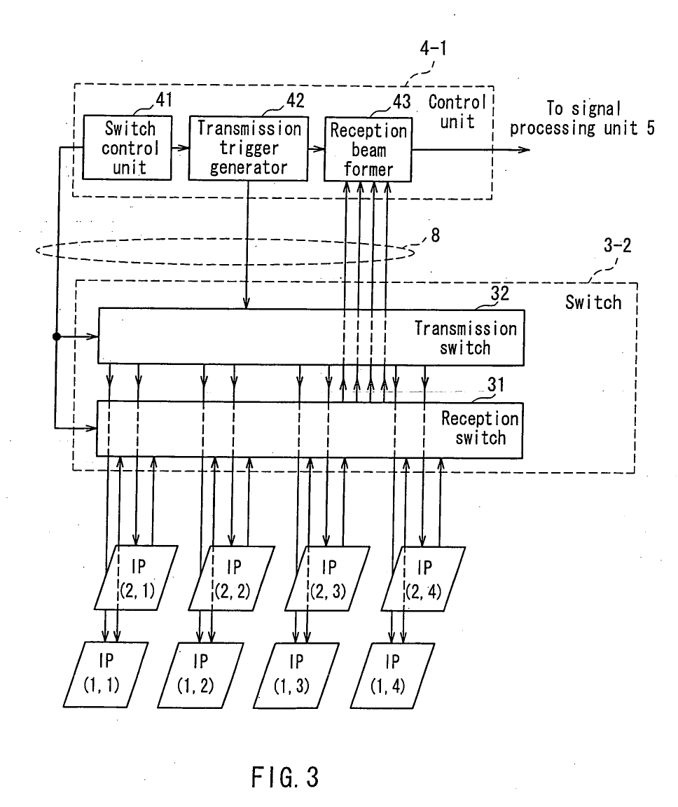

FIG. 3 is a block diagram showing one example of the configuration of a major part of an ultrasonic diagnostic apparatus according to Embodiment 2 of the present invention. In FIG. 3, the same reference numerals and symbols are assigned to the elements having the same configuration and the same functions as those in FIG. 1 referred to in Embodiment 1 so as to omit the explanation therefor. The other elements that are not illustrated in FIG. 3 are the same as in FIG. 1.

In FIG. 3, a switch 3-2 (selection means) includes a reception switch 31 and a transmission switch 32, and a control unit 4-1 includes a switch control unit 41, a transmission trigger generator 42 and a reception beam former 43. The switch 3-2 and the control unit 4-1 are connected via a cable 8. The reception switch 31 and the transmission switch 32 are connected with intra-group processors IP(JJ, KK) (JJ=1 to 2, KK=1 to 4) with J rows (J=2) and K columns (K=4).

The following describes an operation of the switch 3-2 and the control unit 4-1 of the ultrasonic diagnostic apparatus configured as above, with reference to FIG. 3.

Firstly, the switch control unit 41 controls the transmission switch 32 to supply a trigger signal output from the transmission trigger generator 42 to intra-group processors of j rows (j=2) and k columns (k=2) of the intra-group processors IP (JJ, KK). The intra-group processors IP supplied with the trigger signal generate a transmission pulse, and the transmission pulse is supplied to transducers of sub-arrays connected with the intra-group processors IP. The transducers of the sub-arrays generate an ultrasonic pulse in the oriented direction so as to receive an echo from a subject. A reception signal from the sub-array undergoes beam-forming by the intra-group processors IP.

The reception switch 31 selects, under the control of the switch control unit 41, beam former output signals of four lines of the intra-group processors IP with two rows and two columns, and supplies them to the reception beam former 43 via four lines in the cable 8. Herein, the reception switch 31 is composed of an analogue switch having J×K pieces of input terminals and j×k pieces of output terminals. In the case where the reception switch 31 is not provided, eight output signal lines for all of the intra-group processors IP have to be connected with the reception beam former 43. According to the present embodiment, however, the number of the output signal lines to be connected with the reception beam former 43 can be reduced to four. The reception beam former 43 delays and adds the reception signals.

As stated above, according to the present embodiment, the provision of the reception switch 31 allows reception signals of eight intra-group processors to be supplied to the reception beam former 43 via four signal lines in the cable 8 so as to perform delaying and adding of the reception signals. Thus, the number of the signal lines included in the cable 8 can be reduced.

### Embodiment 3

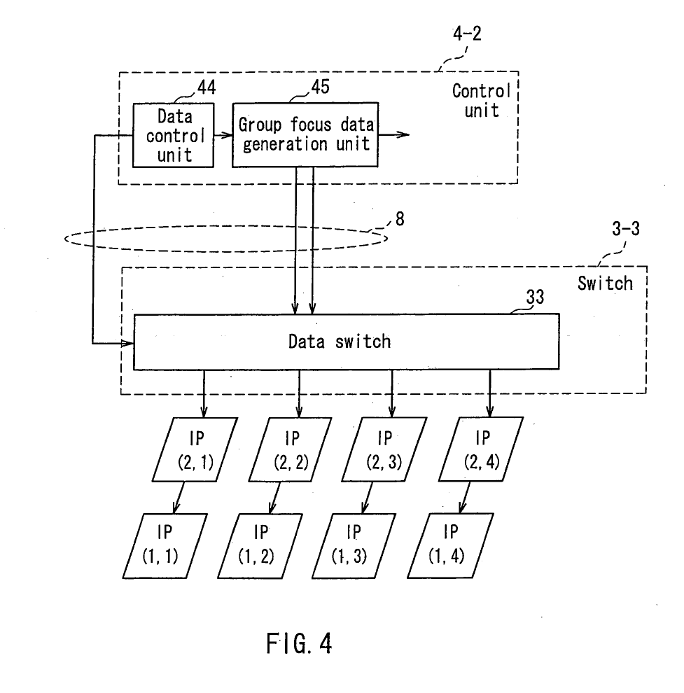

FIG. 4 is a block diagram showing one example of the configuration of a major part of an ultrasonic diagnostic apparatus according to Embodiment 3 of the present invention. In FIG. 4, the same reference numerals and symbols are assigned to the elements having the same configuration and the same functions as those in FIG. 1 referred to in Embodiment 1 so as to omit the explanation therefor. The other elements that are not illustrated in FIG. 4 are the same as in FIG. 1.

In FIG. 4, a switch 3-3 (selection means) includes a data switch 33 and a control unit 4-2 includes a data control unit 44 and a group focus data generation unit 45. The switch 3-3 and the control unit 4-2 are connected via a cable 8. Intra-group processors IP(JJ, KK) (JJ=1 to 2, KK=1 to 4) are connected with the data switch 33. Note here that the data switch 33 in this example has one input and four outputs and data of the group focus data generation unit 45 is output to two adjacent outputs. Although not illustrated, the switch 3-3 includes a transmission switch or a reception switch as in the configuration shown in FIG. 3, which enables the control of communication between the intra-group processors and a reception beam former or a transmission trigger generator of the control unit.

The following describes an operation of the switch 3-3 and the control unit 4-2 of the ultrasonic diagnostic apparatus configured as above, with reference to FIG. 4.

Firstly, the group focus data generation unit 45 generates data that is required for generating ultrasonic pulses or performing beam-forming of reception signals in the intra-group processors. The data generated by the group focus data generation unit 45 is sent to the data switch 33, and the data is supplied, under control of the data control unit 44, to intra-group processors with two rows and two columns that are selected from the intra-group processors IP(JJ, KK). At this time, the data for the intra-group processor IP(1, KK) is supplied via the intra-group processor IP(2, KK).

In the case where the data switch 33 is not provided, data has to be supplied to all of the eight intra-group processors IP. According to the present embodiment, however, it is sufficient to supply data to four intra-group processors IP.

As stated above, according to the present embodiment, the provision of the data switch 33 allows the generation of ultrasonic pulses in selected intra-group processors IP only and the supply of data required for performing beam-forming of reception signals in the selected intra-group processors IP only. Thereby, as compared with the case where data is to be supplied to all of the intra-group processors IP, the amount of data can be reduced and the time to transfer data can be shortened.

### Embodiment 4

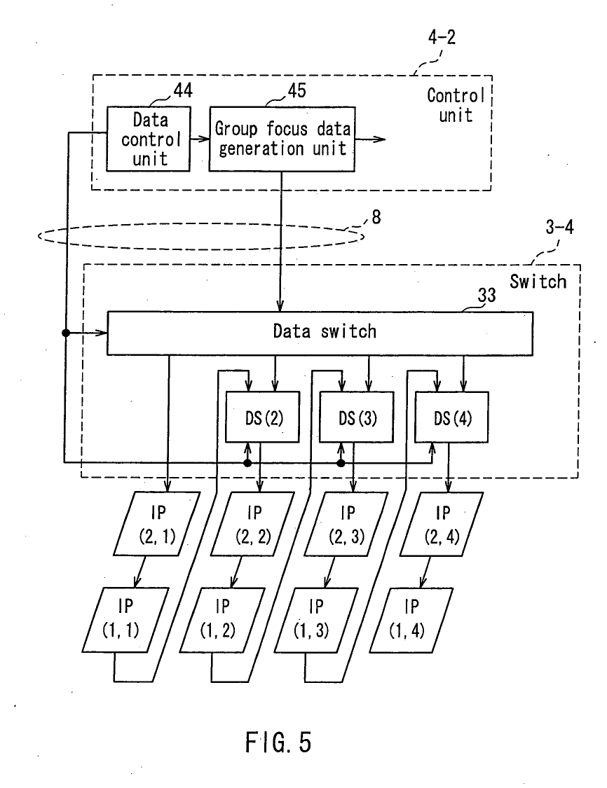

FIG. 5 is a block diagram showing one example of the configuration of a major part of an ultrasonic diagnostic apparatus according to Embodiment 4 of the present invention. In FIG. 5, the same reference numerals and symbols are assigned to the elements having the same configuration and the same functions as those in FIG. 4 referred to in Embodiment 3 so as to omit the explanation therefor. The other elements that are not illustrated in FIG. 5 are the same as in FIG. 1.

In FIG. 5, a switch 3-4 includes a data switch 33 and data selectors DS(I) (I=2 to 4), and a control unit 4-2 includes a data control unit 44 and a group focus data generation unit 45. The switch 3-4 and the control unit 4-2 are connected with a cable 8. Intra-group processors. IP(JJ, KK) (JJ=1 to 2, KK=1 to 4) are connected with the data switch 33 or the data selectors DS(I) (I=2 to 4). Note here that although not illustrated, the switch 3-4 includes a transmission switch or a reception switch as in the configuration shown in FIG. 3, which enables the control of communication between the intra-group processors and a reception beam former or a transmission trigger generator of the control unit.

The following describes an operation of the switch 3-4 and the control unit 4-2 of the ultrasonic diagnostic apparatus configured as above, with reference to FIG. 5.

Firstly, the group focus data generation unit 45 generates data that is required for generating transmission pulses or performing beam-forming of reception signals in the intra-group processors. The data generated by the group focus data generation unit 45 is sent to the data switch 33, and the data is supplied, under control of the data control unit 44, to intra-group processors with two rows and two columns selected from the intra-group processors IP(JJ, KK).

At this time, in the case where the intra-group processors of JJ=1 to 2, KK=1 to 2 are selected from the intra-group processors IP(JJ, KK), the data is supplied by way of a path through the intra-group processors IP (2, 1) and IP (1, 1), the data selector DS (2) and the intra-group processors IP(2, 2) and IP (1, 2).

In the case where the intra-group processors of JJ=1 to 2, and KK=2 to 3 are selected from the intra-group processors IP(JJ, KK), the data is supplied by way of a path through the data selector DS(2), the intra-group processors IP (2, 2) and IP(1, 2), the data selector DS(3), and the intra-group processors IP(2, 3) and IP (1, 3).

In the case where the data switch 33 and the data selector DS(I) are not provided, data has to be supplied to all of the intra-group processors IP. According to the present embodiment, however, this can be simplified by supplying data to four intra-group processors IP.

As stated above, according to the present embodiment, the provision of the data switch 33 and the data selectors DS(I) allows the generation of transmission pulses in selected intra-group processors IP only and the supply of data required for performing beam-forming of reception signals in the selected intra-group processors IP only. Thereby, as compared with the case where data is to be supplied to all of the intra-group processors IP, the amount of data can be reduced and the time to transfer data can be shortened.

### Embodiment 5

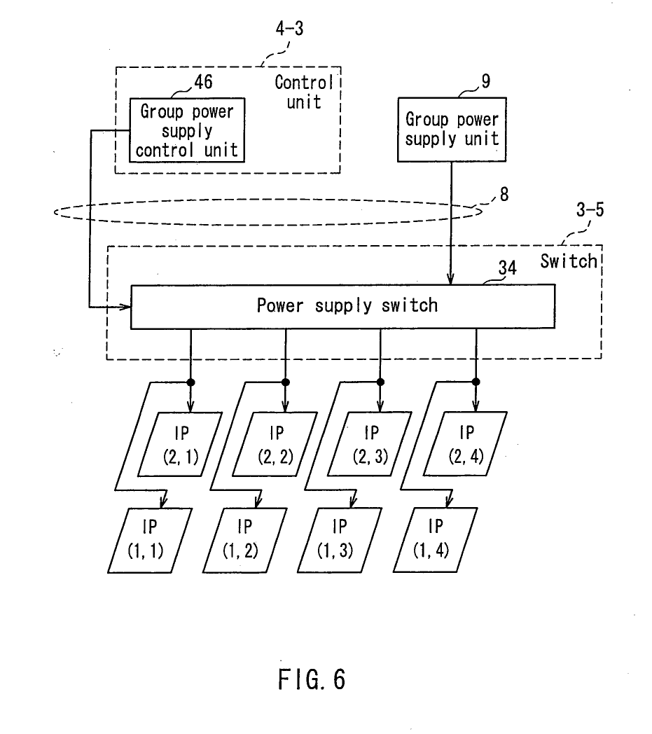

FIG. 6 is a block diagram showing one example of the configuration of a major part of an ultrasonic diagnostic apparatus according to Embodiment 5 of the present invention. In FIG. 6, the same reference numerals and symbols are assigned to the elements having the same configuration and the same functions as those in FIG. 1 referred to in Embodiment 1 so as to omit the explanation therefor. The other elements that are not illustrated in FIG. 6 are the same as in FIG. 1.

In FIG. 6, a switch 3-5 (selection means) includes a power supply switch 34, and a control unit 4-3 includes a group power supply control unit 46. The switch 3-5 is connected with the control unit 4-3 and a group power supply unit 9 via a cable 8. Intra-group processors IP(JJ, KK) (JJ=1 to 2, KK=1 to 4) are connected with the power supply switch 34. Note here that although not illustrated, the switch 3-5 includes a transmission switch or a reception switch as in the configuration shown in FIG. 3, which enables the control of communication between the intra-group processors and a reception beam former or a transmission trigger generator of the control unit.

The following describes an operation of the switch 3-5 and the control unit 4-3 of the ultrasonic diagnostic apparatus configured as above, with reference to FIG. 6.

Firstly, the group power supply unit 9 generates a voltage required for an operation of the intra-group processors. The power supply switch 34 supplies the voltage to intra-group processors selected under control of the group power supply control unit 46. The intra-group processors supplied with the voltage can generate transmission pulses or can perform beam-forming of reception signals. Since no voltage is supplied to the not-selected intra-group processors, no power is consumed by them.

As stated above, according to the present embodiment, the provision of the power supply switch 34 allows the generation of transmission pulses or the beam-forming of reception signals in the intra-group processors supplied with a voltage. Therefore, since no voltage is supplied to the not-selected intra-group processors, their power consumption can be eliminated, thus reducing power consumption as a whole.

### Embodiment 6

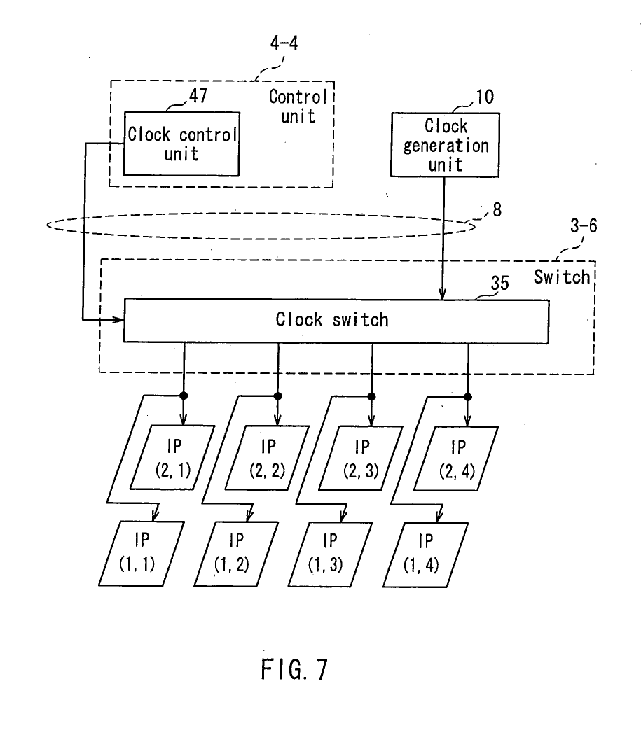

FIG. 7 is a block diagram showing one example of the configuration of a major part of an ultrasonic diagnostic apparatus according to Embodiment 6 of the present invention. In FIG. 7, the same reference numerals and symbols are assigned to the elements having the same configuration and the same functions as those in FIG. 1 referred to in Embodiment 1 so as to omit the explanation therefor. The other elements that are not illustrated in FIG. 7 are the same as in FIG. 1.

In FIG. 7, a switch 3-6 (selection means) includes a clock switch 35, and a control unit 4-4 includes a clock control unit 47. The switch 3-6 is connected with the control unit 4-4 and a clock generation unit 10 via a cable 8. Intra-group processors IP(JJ, KK) (JJ=1 to 2, KK=1 to 4) are connected with the clock switch 35. Note here that although not illustrated, the switch 3-6 includes a transmission switch or a reception switch as in the configuration shown in FIG. 3, which enables the control of communication between the intra-group processors and a reception beam former or a transmission trigger generator of the control unit.

The following describes an operation of the switch 3-6 and the control unit 4-4 of the ultrasonic diagnostic apparatus configured as above, with reference to FIG. 7.

Firstly, the clock generation unit 10 generates clock signals required for an operation by the intra-group processors. The clock switch 35 supplies the clock signals to intra-group processors selected under the control of the clock control unit 47. The intra-group processors supplied with the clock signals can generate transmission pulses or can perform beam-forming by using delay devices that delay receptions signals in accordance with the clock signals. Since no clock signal is supplied to the not-selected intra-group processors, the circuit does not operate partially, thus reducing power consumption.

As stated above, according to the present embodiment, the provision of the clock switch 35 allows the generation of transmission pulses in the intra-group processors supplied with clock signals or the beam-forming of reception signals in the intra-group processors supplied with dock signals. Therefore, since no clock signal is supplied to the not-selected intra-group processors, power consumption can be reduced.

### INDUSTRIAL APPLICABILITY

The ultrasonic diagnostic apparatus of the present invention has advantages of reducing the number of cables for connecting a control unit in a main unit with intra-group processors, reducing transfer time of data supplied to the intra-group processors and reducing power consumption of the intra-group processors. This apparatus is useful as an ultrasonic diagnostic apparatus having two-dimensionally arranged transducers for scanning a subject three-dimensionally, and is applicable to medical use.

1. An ultrasonic diagnostic apparatus, comprising:
an electroacoustic conversion unit including electroacoustic conversion devices with M rows and N columns, in which sub-arrays are arranged at least two-dimensionally with J rows and K columns, each of the sub-arrays including electroacoustic conversion devices with m rows and n columns, where M=m×J, N=n×K;
intra-group processors with J rows and K columns provided corresponding to the respective sub-arrays; and
a selection unit that selects intra-group processors with j rows (j≦J) and k columns (k<K) as a target from the intra-group processors with J rows and K columns, the selection being performed while shifting the selection target of the intra-group processors in a column direction.

2. The ultrasonic diagnostic apparatus according to claim 1, wherein the selection unit selects intra-group processors with j rows and k columns as the target while shifting the selection target of the intra-group processors in a row direction.

3. The ultrasonic diagnostic apparatus according to claim 1, wherein the selection unit comprises a reception switch that selectively connects a reception signal from the intra-group processors with a reception beam former.

4. The ultrasonic diagnostic apparatus according to claim 1, wherein the selection unit comprises a data switch that selectively supplies group focus data to the intra-group processors.

5. The ultrasonic diagnostic apparatus according to claim 1, wherein the selection unit comprises a power supply switch that selectively supplies a group power supply to the intra-group processors.

6. The ultrasonic diagnostic apparatus according to claim 1, wherein the selection unit comprises a clock switch that selectively supplies a clock signal to the intra-group processors.
